(19) United States
(12) Patent Application Publication
(10) Pub. No.: US 2019/0358079 A1
(43) Pub. Date: Nov. 28, 2019

(54) DEVICES, SYSTEMS, AND METHODS FOR TREATMENT OF SLEEP APNEA

(71) Applicants: Ghassan S. Kassab, La Jolia, CA (US); Ali Dabiri, San Diego, CA (US)

(72) Inventors: Ghassan S. Kassab, La Jolia, CA (US); Ali Dabiri, San Diego, CA (US)

(21) Appl. No.: 16/421,404

(22) Filed: May 23, 2019

Related U.S. Application Data
(60) Provisional application No. 62/675,398, filed on May 23, 2018.

(51) Int. Cl. A61F 5/56; A61M 16/04

(52) U.S. Cl. CPC A61F 5/56; A61M 16/0461

(57) ABSTRACT

An assembly for treating sleep apnea comprising a perforated tube comprising a proximal flare and an expandable distal flute and a perforated outer sheath slidably disposed over said perforated tube and having a second proximal flare and comprising a length shorter than the perforated tube. The distal flute may be goblet shaped or spiral shaped such that it is resistant to collapse of soft tissue at the oropharyngeal area of a patient. The assembly may be deployed through the nostril of a patient.

### PRIORITY

The present patent application is related to, and claims the priority benefit of, U.S. Provisional Patent Application Ser. No. 62/675,398, filed on May 23, 2018, the contents of which are hereby incorporated directly and by reference in their entirety into this disclosure.

### BACKGROUND

Fifty to seventy million Americans have sleep or wakefulness disorders, and sleep apnea (SA) affects more at least twelve to eighteen million Americans every year. It is most common among adults over forty-five who are overweight, especially men, but can also affect women, people of normal weight, and even children. Men are twice as likely to have sleep apnea as women. Women are at high risk of SA too if they become obese, post-menopausal, or drink excessive alcohol and smoke.

It is estimated that four out of every hundred middle-aged men and two out of every hundred middle-aged women have obstructive sleep apnea that causes noticeable symptoms. Studies show that sleep apnea occurs in about 2% of children and can occur even in very young children, especially if they are overweight. People who are obese have been found to have four times the risk of developing sleep apnea than people who are at normal weight. Pauses in breathing associated with sleep apnea may last for 10 seconds to one minute and can occur dozens of times per hour in severe SA.

Untreated severe SA can cause high blood pressure and other cardiovascular disease, memory problems, weight gain, impotence, and headaches. Moreover, untreated sleep apnea may, be responsible for job impairment and motor vehicle crashes. There are studies that indicate SA is associated with double the risk of having a stroke.

There are several causes and types of sleep apnea. The main types of sleep apnea are 1) obstructive sleep apnea, which is the more common form that occurs when throat muscles relax (70-80% of the cases), 2) central sleep apnea, which occurs when the brain does not send proper signals to the muscles that control breathing, and 3) complex sleep apnea syndrome (also known as treatment-emergent central sleep apnea), which occurs when someone has both obstructive sleep apnea and central sleep apnea. Obstructive sleep apnea occurs when the muscles in the back of the throat relax. These muscles support the soft palate, the triangular piece of tissue hanging from the soft palate (uvula), the tonsils, the side walls of the throat and the tongue. When the muscles relax, the airway narrows or closes as one breathes in, and hence cannot inspire. This may lower the level of oxygen in the blood and lead to hypoxia. The brain senses this inability to breathe and produces adrenaline to increase heart rate and arouses the person from sleep to reopen the airway. This awakening may be so brief that one does not remember depending on the severity of SA. The obstruction causes pressure fluctuations that lead to snoring, choking or gasping sounds. This pattern can repeat itself five to 30 times or more each hour, throughout the night. These disruptions impair the ability to reach the desired deep, restful phases of sleep, and leads to feelings of sleepiness during waking hours. People with obstructive SA may not be aware that their sleep was interrupted. In fact, some people with this type of SA think they sleep well. Snoring is more than an annoying noise people make when they sleep. The harsh, low-pitched sound comes from the upper airway when it is partially blocked. The flow of air causes tissue in the back of the throat and mouth to vibrate. The noise then comes through the nose, mouth or both the nose and mouth.

There are several possible and conventional treatments for obstructive sleep apnea syndrome (OSAS), including: 1) Positive Airway Pressure (PAP) Therapy, 2) Oral Pressure Therapy, 3) Oral Appliances, 4) Upper Airway Surgery, 5) Hypoglossus Nerve Stimulation, and 6) Behavioral Treatments. Positive airway pressure (PAP) is the first-line of therapy for patients with moderate or severe SCAS. A small blower delivers air pressurized to 5-20 cm H₂O to the upper airway via a nasal mask, full-face mask. The air pressure prevents the upper airway from collapsing during sleep. This method of treatment is highly effective and has enormous health benefits when the PAP device is used consistently and as recommended. This therapy dramatically improves the quality of sleep, reduces Excessive Daytime Sleepiness (EDS), improves mood and function, and decreases the incidence of motor vehicle accidents.

Continuous positive airway pressure (CPAP) devices deliver a single fixed pressure during both inhalation and exhalation. Some patients find it hard to sleep while using the CPAP device, however, and initial acceptance rates are only around 70%. Long-term adherence can be challenging, and studies demonstrate that up to 50% of patients who have initially accepted CPAP will not wear the apparatus for a full night of sleep. Adherence is lowest for individuals with mild OSAS, compared to those with more severe disease, because they derive least subjective benefit from CPAP.

Bilevel positive airway pressure (BPAP) delivers a higher air pressure during inhalation (inspiratory positive airway pressure [IPAP] and a lower expiratory positive airway pressure [EPAP]). The BPAP device can sense a patient's efforts to exhale and drops the air pressure accordingly. This mode of PAP therapy may be useful for patients who have difficulty exhaling against the air pressure of traditional CPAP, require high PAP settings, or develop gastric distention from swallowing air while on CPAP therapy.

Autotitrating positive airway pressure (APAP) devices provide the same pressure during inhalation and exhalation, but the setting can vary during sleep, depending on the presence or absence of apneas, hypopneas, or snoring. This minimizes the average overnight pressure, and may improve patient adherence. In addition, APAP can be used to initially establish therapeutic pressure, avoiding a second night in the sleep laboratory.

Oral Pressure Therapy (OPT) is a proprietorial treatment for OSAS that does not use a mask. Instead, the system uses a mouthpiece fitted to the specific individual, a small vacuum console, and flexible tubing that connects the two. The system applies a light vacuum to reposition the tongue and soft palate, thereby opening the sleeper's airway and reduces OSAS. The benefits of this system are that the individual can easily sleep in any position and does not have to wear a mask. You need to have a vacuum device to operate the unit.

Oral Appliances move the tongue or lower jaw forward and upward, which increases the size of the upper airway, and helps the patient breath. These devices look like mouth guards that should always be fitted by a dentist who is trained in sleep medicine. These devices are mainly used for the treatment of patients with snoring or mild to moderate OSAS, and those for whom CPAP therapy has been unsuccessful.

Upper Airway Surgery can be considered for patients with anatomical facial abnormalities that contribute to obstructed airways, maxillary-mandibular advancement may be considered to improve upper airway space. In severely overweight or obese patients, bariatric surgery may be considered as part of a multi-disciplinary approach to assist the patient with weight loss efforts. Limited information exists on the long-term outcomes of surgery with respect to OSAS. The degree of success depends on the anatomy of the upper airway and on the type of surgery.

Regarding Hypoglossus Nerve Stimulation (HGNS), and in 2014, the Food and Drug Administration (FDA) approved a new OSAS treatment using hypoglossus nerve stimulation, which is targeted at adults with moderate to severe OSAS who cannot be treated successfully with other treatments. The treatment is indicated in such patients who have a body mass index (BMI)<32, and in whom drug induced sleep endoscopy excludes concentric obstruction of the airway during sleep. The system is a nerve stimulator that is implanted in the patient's chest; leads are connected to the hypoglossal nerve (cranial nerve XII), which controls tongue movement, and to a breathing sensor. The system monitors the patient's breathing patterns and stimulates the hypoglossal nerve during inhalation in order to maintain an open airway and minimize OSAS. The patient operates the system using a remote control that is activated before going to sleep and that deactivates upon waking. The overall rate of serious adverse events was <2%, and 98% of participants were still using the system at the end of the 12 months. The disadvantage of the system is cost and the need for surgery.

Behavioral Treatments may be successful in the milder forms of OSAS and primarily involve such lifestyle modifications as weight loss and limiting the use of alcohol or sedatives. In patients with positional OSAS (for whom OSAS is only present when the patient sleeps in a supine position), devices may be used to help the person reposition him or herself and minimize the OSAS. For example, there are several types of wearable sensors that use vibrations to train the individual not to sleep on his or her back, thereby minimizing OSAS symptoms.

These devices have shortcomings ranging from lack of efficacy to bulky mechanisms to significant costs. In view of the foregoing, there is a need for devices, systems, and methods to treat obstructive SA which eliminates the negative impacts of conventional treatments, and said devices, systems, and methods would be well received in the marketplace. The Aspiration Relief System (ARS), notably exemplary devices of the present disclosure, targets patient population suffering from mild to moderate OSA. The proposed device is novel in the following respects: 1) Minimally invasive approach; i.e., catheter based; 2) No moving parts or noise generation; and 3) Lightweight, simple to use and cost effective.

In view of the foregoing, there is a need for devices, systems, and methods to treat obstructive SA which eliminates the negative impacts of conventional treatments, and said devices, systems, and methods would be well received in the marketplace.

### BRIEF DESCRIPTION

In an exemplary embodiment an assembly for treating sleep apnea comprises a tube comprising an expandable distal flute; and an outer sheath having a length shorter than the perforated tube and slidably disposed over said tube.

In a further embodiment, the tube and outer sheath are perforated. The assembly may comprise an undeployed position where the sheath is disposed over the distal flute such that the distal flute is in a compressed position. The tube and outer sheath may each comprise a proximal flare. The outer sheath may comprise a tapered tip. The distal flute may be expandable radially. The flute may be goblet shaped. The distal flute may comprise generally longitudinal prongs or comprise a spring or coil shape. The outer sheath may be is 5 mm-2 cm shorter than the tube.

In another embodiment, an assembly for treating sleep apnea comprises a perforated tube comprising a proximal flare and an expandable distal flute; and a perforated outer sheath slidably disposed over said perforated tube and having a second proximal flare and comprising a length shorter than the perforated tube.

The proximal flare of the tube may comprise a proximal diameter larger than a proximal diameter of the proximal flare of the sheath so that the sheath cannot move proximally beyond the tube. The proximal diameter of the proximal flare of the tube may be larger than the nostril of a patient.

In an assembly for treating sleep apnea, the outer sheath is 5 mm-2 m shorter than the tube. In an assembly for treating sleep apnea, the outer sheath is 1 mm larger in diameter than the tube. In an assembly for treating sleep apnea, the tube is 2-3 mm in diameter.

An exemplary embodiment of a method of using an assembly for treating sleep apnea comprises: inserting the assembly into the nostril of a patient wherein the assembly comprises: a tube comprising an expandable distal flute; and advancing the assembly such that the distal flute is disposed into the oropharyngeal area of the patient; and expanding the distal flute.

In a further embodiment, a method of using an assembly for treating sleep apnea, wherein the assembly further comprises an outer sheath slidably disposed over the tube, further comprises the step of moving the outer sheath proximal relative to the tube.

In a further embodiment, a method of using an assembly for treating sleep apnea, a method of using an assembly for treating sleep apnea further comprises the step of holding the nasal passage open.

A method of using an assembly for treating sleep apnea, the assembly further comprises an outer sheath having a tapered distal tip and the method further comprises the step of lubricating the distal tip of the sheath.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1A shows a PPT configuration before insertion;

FIG. 1B shows a PPT configuration after insertion and moving the outer sheath to expose the goblet or flared shaped flute according to an exemplary embodiment of the present disclosure;

FIG. 2 shows a similar device as shown in FIGS. 1A and 1B except that the flute is spring shaped, according to an exemplary embodiment of the present disclosure;

FIG. 3 shows the schematic of PPT and its assembled position in the upper airway down to oropharynx region, according to an exemplary embodiment of the present disclosure;

FIG. 4 shows the anatomy of nasal cavity, pharynx, larynx, epiglottis, and tongue to show the oropharynx region, according to an exemplary embodiment of the present disclosure;

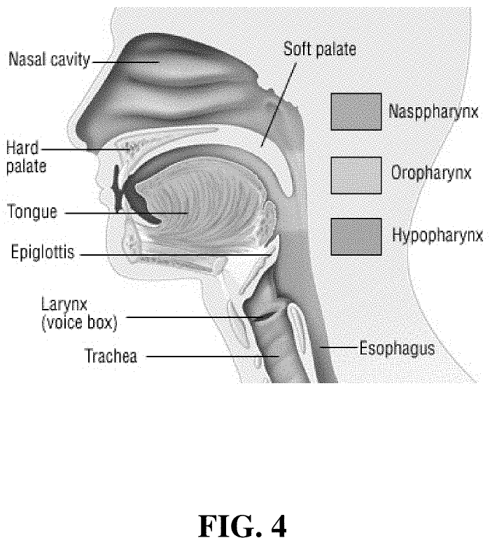

FIG. 5 shows the PPT in its assembled position, according to an exemplary embodiment of the present disclosure; and

FIG. 6 shows the schematic of the PPT inside of the obstructive airway, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

A primary objective of the devices, methods and apparatuses of the present disclosure is to provide systems and methods for treating the obstructive SA which eliminates the issues of the conventional treatments.

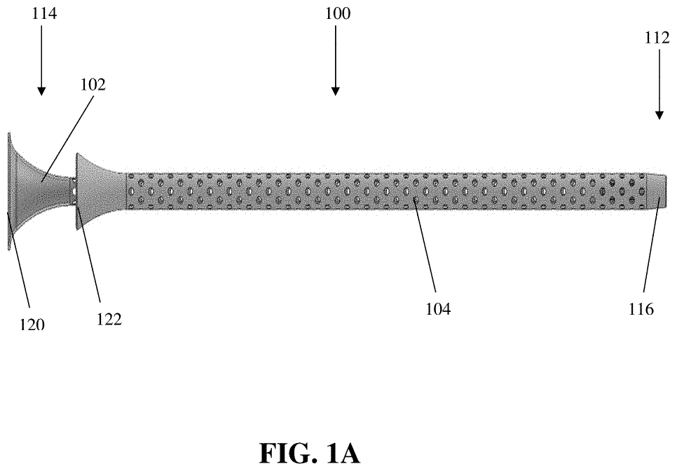

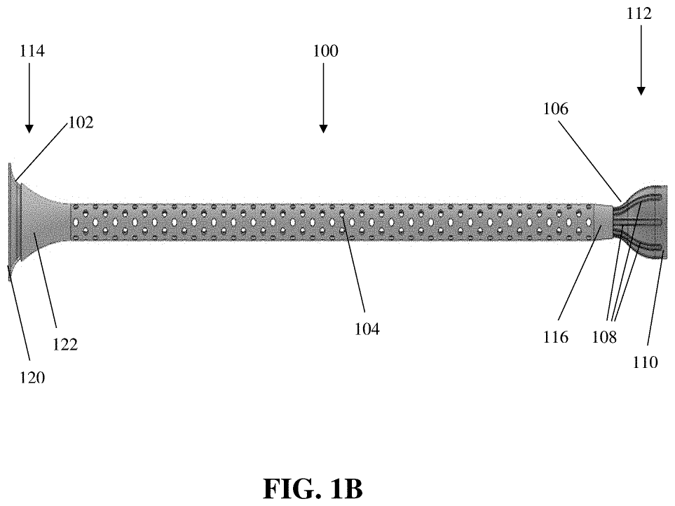

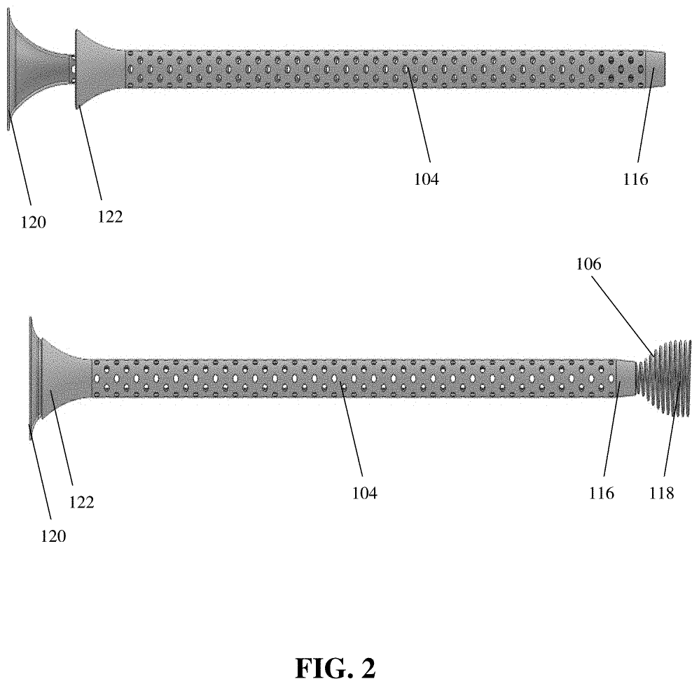

An exemplary device of the present disclosure comprises the assembly 100. The assembly 100 comprises the biocompatible perforated plastic tube (PPT) 102 and an outer sheath 104, such as according to the configurations shown in FIGS. 1A, 1B, and 2.

The PPT 102 can be housed in a biocompatible outer sheath 104, wherein the sheath 104 is slidably engaged with and disposed over the PPT 102. The PPT 102 may comprise a flute 106 at the distal end 112 of the assembly 100. As shown in FIG. 1A-B, the flute 106 can be goblet shaped for improved support, and can be made from a multi-pronged 108 nitinol (or other biocompatible material) frame that expands when deployed. In this embodiment, the prongs 108 extend longitudinally and expand radially outward. The flute 106 can be bare or coated with an elastomer 110 as shown in FIGS. 1A and 1B, for example. In the embodiment of FIG. 2, the flute 106 can also be spring or coil 118 shaped such that it would expand radially when it deployed. The length of the PPT 102 should be tailor sized from the entrance of the patient's nose to the patient's throat using imaging (cone beam x-ray, CT, etc.) data of patient. Other embodiments of the PPT may use a material other than plastic.

The sheath 104 may also be perforated. The length of the sheath 104 will be shorter than the length of the PPT 102, preferably around 5 mm-2 cm. In an undeployed configuration, the sheath 104 is disposed toward the distal end of the assembly 100 and the sheath 104 acts as a sleeve to squeeze the flute 106 compressed. The distal end of the outer sheath 104 has a tapered tip 116 for smooth movement through the nose. This assembly 100 can be gently inserted through the nose, past the upper airway, and down to the throat where the muscles in the back of the throat relax. The proximal ends 114 of the PPT 106 and outer sheath have flares 120, 122 for improved breathing while the distal end provides a flute 106 that resists any collapse of soft tissue. The diameter of the flare 120 of the tube will be larger than the diameter of the flare 122 of the sheath so that the sheath 104 cannot move proximally beyond the tube. Both the sheath and the PPT are flexible.

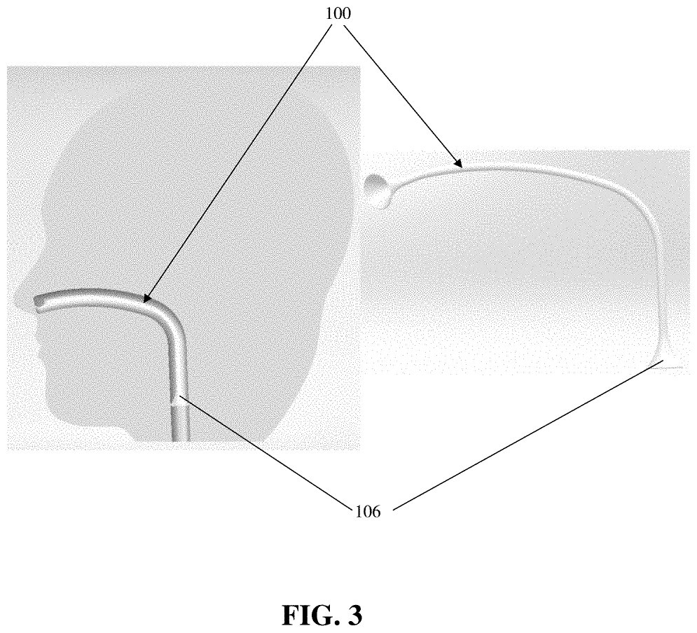

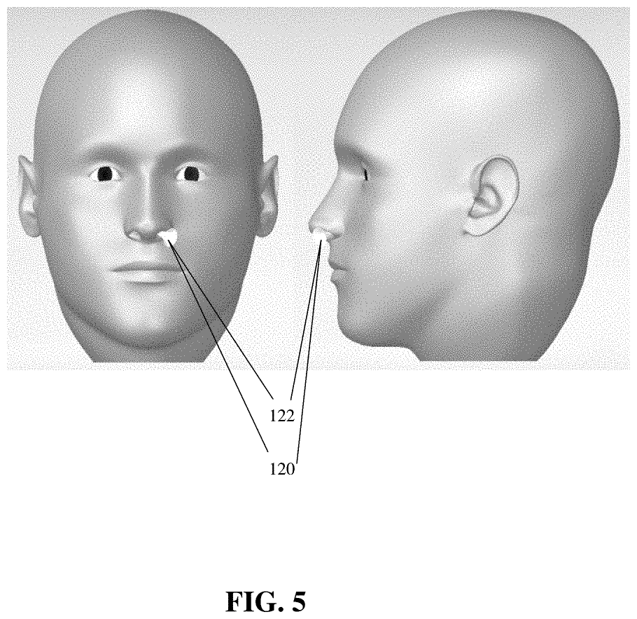

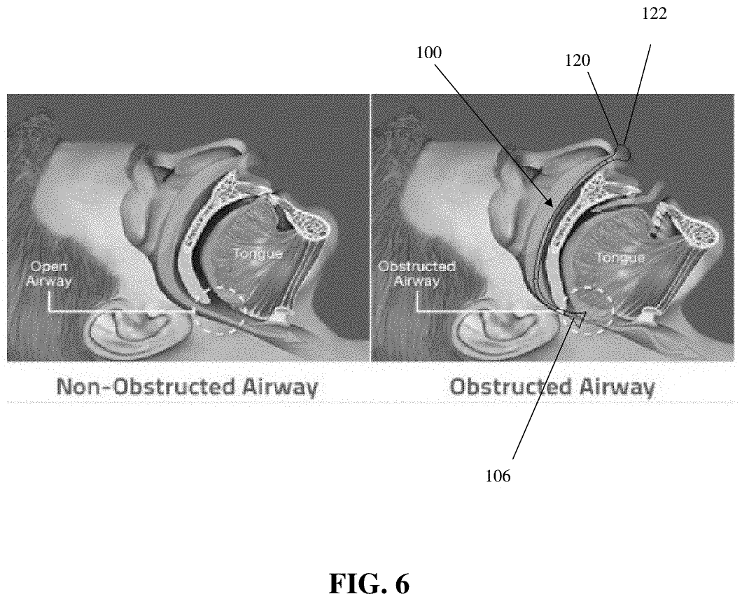

The patient needs to be in the upright position during insertion of the assembly 100. This is the location where the airway narrows or closes as the patient inspires in the area of oropharynx according to FIG. 3. The tip of the tube will be lubricated device to make sure that the patient will not be uncomfortable during the PPT 102 insertion. The assembly 100 is advanced through the patient's nasal cavity and to the oropharynx area. Once the assembly 100 is in place, the outer sheath 104 will be moved proximally relative to the PPT 102, to the deployed position. This displaces the sheath 104 just a short distance (5 mm-2 cm) to expand the flute 106. The flute 106 will keep the passage close to oropharynx open during the sleep and the proximal flaring will stop the PPT 102 from going further down the throat. The proximal flaring needs to be longer to compensate for the outer sheath 104 displacement and at the same time keep the PPT 102 from going further down the throat. The diameter of the PPT 102 is 2-3 mm in several embodiments, and the diameter of the sleeve is larger than the diameter of the PPT 102 by about 1 mm or so to ensure smooth operation. The breathing air will pass through inside the PPT 102 as well the outside of the PPT 102. The purpose of the perforation is to make sure that the breathing air is subject to filtration of dirt, humidification and thermalization before it reaches the lung as is normally the case. FIG. 5 shows the PPT 102 in the assembled position which shows how it is stopped by the proximal flaring 120, 122. FIG. 6 shows schematic of the PPT 102 inside of the obstructive airway. In the morning, the patient will return the sleeve to its original position before withdrawal of the assembly 100. The assembly 100 will be rinsed in a solution before storage for reuse.

The advantage of this device is the simplicity of operation and cost. It would take the patient about a week to get used to the procedure. The device is reusable and can be sanitized and dried day after day. It does not have the drawback of the devices in the market.

While various embodiments of devices, systems, and methods have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. An assembly for treating sleep apnea comprising:
a tube comprising an expandable distal flute; and
an outer sheath having a length shorter than the perforated tube and slidably disposed over said tube.

2. The assembly of claim 1, wherein the tube and outer sheath are perforated.

3. The assembly of claim 2, comprising:
an undeployed position where the sheath is disposed over the distal flute such that the distal flute is in a compressed position.

4. The assembly of claim 3 wherein the tube and outer sheath each comprise a proximal flare.

5. The assembly of claim 4, wherein a distal end of the outer sheath comprises a tapered tip.

6. The assembly of claim 5, wherein the distal flute is expandable radially.

7. The assembly of claim 6, wherein distal flute comprises generally longitudinal prongs

8. The assembly of claim 6, wherein the distal flute comprises a spring or coil shape.

9. The assembly of claim 1, wherein the outer sheath is 5 mm-2 cm shorter than the tube.

10. An assembly for treating sleep apnea comprising:
a perforated tube comprising a proximal flare and an expandable distal flute; and
an perforated outer sheath slidably disposed over said perforated tube and having a second proximal flare and comprising a length shorter than the perforated tube.

11. The assembly of claim 10, wherein the proximal flare of the tube comprises a proximal diameter larger than a proximal diameter of the proximal flare of the sheath so that the sheath cannot move proximally beyond the tube.

12. The assembly of claim 10, wherein the flute is goblet shaped.

13. The assembly of claim 11, wherein the proximal diameter of the proximal flare of the tube is larger than the nostril of a patient.

14. The assembly of claim 10, wherein the outer sheath is 5 mm-2 cm shorter than the tube.

15. The assembly of claim 10, wherein the outer sheath is 1 mm larger in diameter than the tube.

16. The assembly of claim 10, wherein the tube is 2-3 mm in diameter.

17. A method of using an assembly for treating sleep apnea comprising:
inserting an assembly into a nostril of a patient, the assembly comprising a tube comprising an expandable distal flute;
advancing the assembly such that the distal flute is disposed into the oropharyngeal area of the patient; and
expanding the distal flute.

18. The method of claim 17, wherein the assembly further comprises an outer sheath slidably disposed over the tube and further comprising the step of moving the outer sheath proximal relative to the tube.

19. The method of claim 17, wherein the step of expanding the distal flute further comprises the step of holding the breathing passage open.

20. The method of claim 17, wherein the assembly further comprises an outer sheath having a tapered distal tip and the method further comprises the step of lubricating the distal tip of the sheath.
